(19) United States
(12) Patent Application Publication
(10) Pub. No.: US 2017/0196488 A1
(43) Pub. Date: Jul. 13, 2017

(54) Serial Electrochemical Measurements of Blood Components

(71) Applicant: SRI INTERNATIONAL, Menlo Park, CA (US)

(72) Inventors: Jonathan Hofius, Menlo Park, CA (US); Jose P. Joseph, Menlo Park, CA (US); Manish Kothari, Menlo Park, CA (US); Pablo E. Garcia Kilroy, Menlo Park, CA (US)

(73) Assignee: SRI INTERNATIONAL, Menlo Park, CA (US)

(21) Appl. No.: 15/468,105

(22) Filed: Mar. 23, 2017

Related U.S. Application Data
(63) Continuation of application No. PCT/US15/51803, filed on Sep. 23, 2015.
(60) Provisional application No. 62/053,978, filed on Sep. 23, 2014.

(51) Int. Cl. A61B 5/1486; A61B 5/00; A61B 5/145

(52) U.S. Cl. CPC A61B 5/1486; A61B 5/14532; A61B 5/14546; A61B 5/6852

(57) ABSTRACT

Devices, systems and methods for measuring, and configured to measure, a blood analyte continuously or at intervals, the device comprising at least a first set of analyte sensing sensor electrodes configured for making electrochemical measurements of the analyte, and at least a second set of biofouling prevention electrodes in operable proximity to, and configured to prevent biofouling of, the first set of electrodes.

This application is a continuation of PCT/US15/51803; filed Sep. 23, 2015, which claims priority to Ser. No. 62/053,978; filed Sep. 23, 2014.

### INTRODUCTION

Lactate levels in blood are an important indicator of the general health of a person. Lactate measurements may be done for various reasons such as to test for hypoxia (lack of blood and oxygen), infectious disease such as HIV, cardiac conditions, shock and sepsis and for management of the same, and for clinical exercise testing as well as during performance testing of athletes.

Ongoing clinical studies are examining the role of serial blood lactate measurements in the management of shock in patients with trauma or sepsis [1]. According to [1], “serial lactate values followed over a period of time can be used to predict impending complications or grave outcome in patients of trauma or sepsis. Interventions that decrease lactate values to normal early may improve chances of survival and can be considered effective therapy. Lactate values need to be followed for a longer period of time in critical patients.”

Current on-market techniques to measure lactate levels for the management of critically ill patients include placement of a central venous catheter (CVC) and taking blood samples for in-vitro testing and analysis. A CVC is essentially a synthetic tube inserted into a patient such that the tip of the CVC lies within the superior vena cava (SVC). The CVC is used to administer fluids, medicines, parenteral nutrition and blood. It may also be used to draw samples of blood so that patients do not need be pricked constantly. CVCs are used not only in hospitals, but also homes, nursing care facilities etc. Generally, the CVCs used in out-of-hospital settings are placed peripherally (i.e. through the arm) and hence these types of CVCs are called Peripherally Inserted Central Catheter (PICC) line.

In addition to in-vitro testing, there have been attempts in research environments to measure levels of blood chemicals with sensors inserted into catheters such as the CVC. However these techniques are not common place due to a myriad of issues including cost, complexity and accuracy of these devices. This disclosure addresses these issues enabling in-vivo measurements in a fast, reliable and cost-effective manner

There are several risks with the use of long term indwelling catheters whether in the hospital or outside of the hospital. One is the risk of thrombosis or blood clots forming around the catheter tips and the sensors used to measure the lactate levels. A discussion of the risks associated with catheter related thrombosis is presented in “Management of occlusion and thrombosis associated with long-term indwelling central venous catheters”[2]. In a situation where a catheter such as a CVC is measuring lactate levels continuously or at certain intervals, a thrombolytic or partially thrombolytic catheter may lead to incorrect lactate level readouts. Current methods to address the clotting include removing catheter and replacing it with a new catheter. Clots may also be dissolved by medication such as Alteplase which may be infused within the catheter which may have side effects such as bleeding. Thus there is a need to address the situation with a sensor that can be integrated with an in-dwelling catheter so that in-vivo continuous or semi-continuous (i.e. at determined, scheduled and/or period intervals) lactate level measurements can be made but where the risk of clotting is minimized or eliminated.

In addition to continuous lactate monitoring, glucose is another parameter that needs to be monitored continuously especially in patients in the intensive care unit. According to [3], “elevated glucose levels in critically ill patients have been shown to be related to increased mortality and length of hospital stay in adults and children. The impact of tight glycemic control on clinical outcomes of patients in the intensive care setting has recently gained recognition”. Also according to [3], two common procedures to measure blood glucose levels are via venous/arterial blood by way of an indwelling vascular catheter and via capillary (finger prick) blood. The authors of [3] state, “Venous/arterial vascular blood sampling is time consuming, carries a risk of infections and complications, and involves a relatively large amount of blood drawn”. Hence, there is a need for a sensor that can be integrated with an in-dwelling catheter so that in-vivo measurements can be made. The risk of clotting remains the same for either case and needs to be minimized or reduced.

Another risk encountered by patients with long term in dwelling catheters is the risk of biofilm formation on the catheter surface or sometimes on the inside walls of the catheter or both (WO2012/177807). Biofilms may be bacterial or fungicidal or both. Biofilms are hard to treat and are sometimes resistant to treatments. Sensors such as the lactate sensor if introduced in the blood stream are prone to biofilm formation in addition to being prone to clot formation. Thus there is a need for sensors that measure blood chemicals such as lactate and glucose in an environment where the sensors are immersed in flowing blood in such a way that the risks of blood clots formation and biofilm formation are reduced or eliminated. While this disclosure emphasizes measuring lactate and glucose, other blood chemicals including but not limited to urea may be also measured.

### SUMMARY OF THE INVENTION

Several approaches to reduce or eliminate clotting are described. One aspect is based on the concept of applying a voltage across two electrodes, which reduced or prevents the formation of thrombus across, on or near the two electrodes. In embodiments the electrodes that measure a blood analyte have another set or sets of electrodes in the near vicinity. To distinguish between the two different types of electrodes, the electrodes that measure the blood chemicals are called sensor or analyte sensing electrodes and the electrodes that prevent blood clots and biofilm formation are called biofouling prevention electrodes. Hence with the biofouling prevention electrodes in close proximity to the sensor electrodes, while the analyte (e.g. lactate or glucose) levels are measured amperometrically with the latter electrodes, the former set or sets of electrodes prevents the formation of clots or biofilms. Further, it has been observed by the authors that the chemical reactions concerning biofouling prevention occurs predominantly on one electrode compared to the reactions at the other electrode. Hence, in embodiments the polarity of the biofouling prevention electrodes is switched at intervals of time which may be periodic or aperiodic. Generally the electrode at which the reactions predominantly occur will be called the “working electrode” whereas the other electrode will be called the “counter electrode”. The working electrode may be the anode but it is not necessary for the working electrode to be connected to a positive terminal of a battery source.

In some approaches, the biofouling prevention electrodes are placed around the sensor electrodes in planar structures. In some other approaches, the sensor electrodes are placed in a pocket and the biofouling prevention electrodes formed in shape of a grid are placed on top of sensor electrodes.

In yet other approaches, methods and systems are described which do not depend on electrochemical dissolution of clots and biofilms. In these approaches a series of sensors is used where only one sensor is exposed to blood at any one time. When a measurement from that sensor is obtained, another sensor is exposed. Several variations of this approach are described below.

In yet more approaches, a system is described where serial glucose or lactate measurements are done right at the patient site in-vitro, and with this system serial measurements can be done very quickly.

In an aspect the invention provide a device or system substantially as disclosed herein, including the drawings.

In an aspect the invention provides a device, typically vein insertable or implantable, comprising: (a) a pair of anode and cathode elongate sensor electrodes, each comprising a distal, terminal tip comprising a surface catalyst which catalyzes a chemical reduction-oxidation (redox) reaction of a blood analyte yielding an amperometric measurement of the analyte; and (b) a pair of anode and cathode elongate antifouling electrodes, each comprising an uninsulated, distal, terminal tip, between which an electrical current flows, wherein the sensor and antifouling electrode tips are disposed on a planar surface, which may be flat or curved, and the antifouling electrode tips sufficiently surround one or both of the sensor electrode tips wherein when disposed in a vein the current causes chemical reactions in the blood around one or both of the sensor electrodes tips which reduces or prevents biofouling of the tip of one or both of the sensor electrodes.

As shown in the drawings, the tip of each electrode is the distal, active portion where the sensing and antifouling effects occur. The tips may be of a wide variety of shapes and configurations, such as shown in the drawings. The elongate structure refers to the electrodes, including the leads and the tips.

In embodiments:

the planar surface is flat;

the sensor electrode tips are disposed on insulator pads.

the device disposed in the lumen of a vein or artery;

the device is disposed in the lumen or on the surface of an implanted catheter;

the sensor electrode tips are separated by 1 nm to 1 mm;

the sensor and antifouling tips are separated by 1 nm to 1 mm; and/or

the sensor electrodes are set in a pocket covered by one of the antifouling electrode tips and patterned as a grid providing the one or more gaps.

In another aspect the invention provides a device, typically vein insertable or implantable, comprising a series of sensors, each sensor comprising a pair of elongate sensor electrodes, each comprising a distal, terminal tip comprising a surface catalyst which catalyzes a chemical reduction-oxidation (redox) reaction of a blood analyte yielding an amperometric measurement of the analyte, wherein the series of sensors is printed on a rotatable strip within a catheter, rotated so that each of the sensors is exposed to blood for a redetermined time sufficiently limited to reduce or prevent biofouling of the tip of one or both of the sensor electrodes.

This aspect includes the foregoing embodiments, and or an embodiment wherein electrical connections to the tips are made via brushes which stay stationary in one place while the strip slides beneath it.

In another aspect the invention provides a device comprising a sensor, capable of measuring, and configured to measure, a blood analyte continuously or at intervals, the device comprising at least a first set of analyte sensing sensor electrodes configured for making electrochemical measurements of the analyte, and at least a second set of biofouling prevention electrodes in operable proximity to, and configured to prevent biofouling of, the first set of electrodes.

This invention also includes the foregoing embodiments and embodiments wherein:
the sensor and biofouling prevention electrodes are elongate, and the sensor and antifouling electrode tips are disposed on a planar surface, which may be flat or curved;
the each of the sensor electrodes of the first set is surrounded by biofouling prevention electrodes of the second set;
the sensor electrodes are set in a pocket covered by a first biofouling prevention electrode of the second set and patterned as a grid with hole size smaller than the size of white blood cells (less than 10 um but larger than 7 um), wherein a second biofouling prevention electrode of the second set is configured in a planar manner to the first electrode;
the sensor electrodes and the biofouling prevention electrodes are located at the distal end of a catheter, inside the lumen of the catheter and/or on the surface of the catheter;
the device is configured for:

switching the polarity of the biofouling prevention electrodes;

measuring an analyte that is lactate or glucose; and/or

preventing biofouling that is clot formation or growth of bacteria or fungi; and/or
the biofouling prevention electrodes are at a distance from the sensor electrodes about or between 1000, 500, 200, 100 or 50 uM and 20, 10, 5, 2 or 1 uM.

In another aspect the invention provides a device or a system configured to be capable of measuring a blood analyte at intervals with a series of sensors arranged: on a strip configured to be inserted into a sheath with an opening such that only one of the series of sensors is exposed to blood at any one time; and/or on a side of a drum which rotate inside the sheath with an opening such that only one of the series of sensors is exposed to blood at any one time.

In embodiments of the device or system:

the sheath and the sensor strip may be inserted into a catheter;

the sensors are arranged in a parallel configuration;

the sensors are arranged so that they come in contact with a brush placed inside the sheath;

each sensor is compartmentalized so that only the sensors not under the opening of the sheath are not contaminated; and/or

comprising multiple different analyte sensors in a single strip, such as lactate and glucose.

In another aspect the invention provides a method of using a subject device or system comprising continually or continuously measuring impedance between the biofouling prevention electrodes and switching on higher voltages when higher impedance is sensed.

In another aspect the invention provides a method of using a subject device or system comprising multiplying the concentration values read by the sensor electrodes by a constant dependent on the impedance between the biofouling prevention electrodes.

The invention specifically provides all combinations of the recited embodiments, as if each had been laboriously individually set forth.

### BRIEF DESCRIPTION OF THE FIGURES

FIG. 1A: Reactions within blood that enable measurement of lactate levels amperometrically.

FIG. 1B: Cross section of the electrode.

FIG. 2A: Disposable lactate sensor.

FIG. 2B: Placement of the lactate sensor within a blood vessel.

FIG. 3A: Relationship between the H2O2 concentration and the induced and increased current due to the disassociation of H2O2 at the working electrode.

FIG. 3B: Relationship between the lactate concentration and the induced but increased current according to Eqn. 1 and Eqn. 2

FIG. 4A: Clotted blood before any electrochemical activation.

FIG. 4B: Clots diminishing after the application of a 20 uA current for 10 mins

FIG. 4C: Upon application of the current for 1 hour, the clot has almost dissolved.

FIG. 5A: Planar configuration of sensor and biofouling catheters.

FIG. 5B: Alternative planar configuration of sensor and biofouling catheters.

FIG. 5C: Configuration where the sensor electrodes are placed in a pocket and where a grid covers the pocket.

FIG. 5D: Side view of configuration where the sensor electrodes are placed in a pocket and where a grid covers the pocket.

FIG. 6A: Sensor with biofouling prevention electrodes placed within a catheter.

FIG. 6B: Shape of sensor bases.

FIG. 6C: Alternative shape of sensor bases.

FIG. 7: Sensor electrodes and biofouling prevention electrodes placed on the outside surface of an implantable catheter.

FIG. 8A: Strip of sensors arranged in a parallel circuit.

FIG. 8B: Strip of sensors with a brush connection.

FIG. 8C: Side view of the brush connection and a sensor electrode.

FIG. 8D: Strip of sensors placed within a sheath inside a catheter.

FIG. 8E: Series of sensors exposed to blood one at a time.

FIG. 8F: Arrangement of sensors which rotate on a drum, also to expose the sensors one at a time to blood.

FIG. 8G: Sensors compartmentalized when a strip of sensors is used.

FIG. 8H: Sensors compartmentalized when a rotatory configuration of FIG. 8F is utilized.

FIG. 9A: Proximal side of a catheter when the sensor configuration in FIG. 6A-C is utilized. This figure also illustrates how the sensor may be electrically connected to an external circuit.

FIG. 9B: Proximal side of a catheter when the sensor configuration in FIG. 8A or 8B is utilized.

FIG. 9C: Configuration of the walls and the sensors for the sensor strip.

FIG. 10A: In-vitro system to make a series of measurements of the levels blood compounds.

FIG. 10B: One cylinder with electrodes used in the system in FIG. 10A.

FIG. 10C: Perspective view of the microanalysis platform of FIG. 10A.

### DETAILED DESCRIPTION OF PARTICULAR EMBODIMENTS AND EXAMPLES THEREOF

In-Vivo Sensor

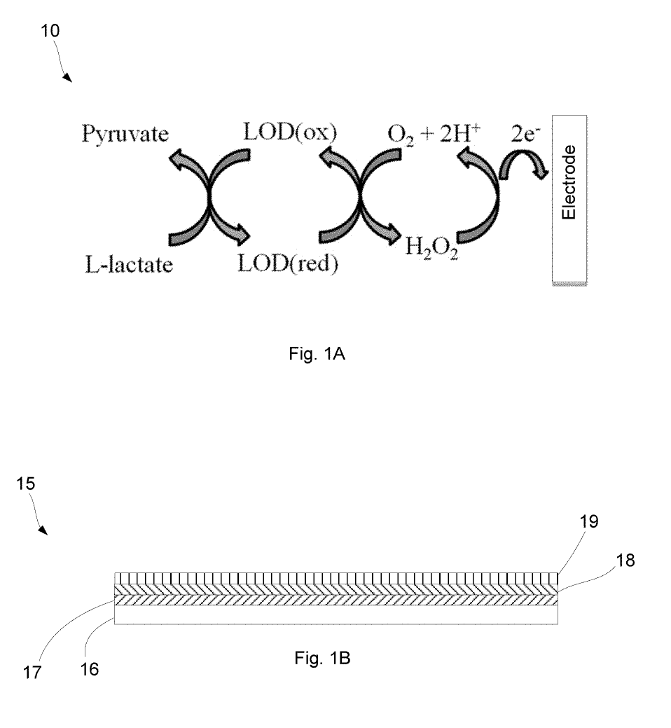

FIG. 1A illustrates the electrochemical process by which blood lactate is measured amperometrically. The basic reactions corresponding to the figure are given below:

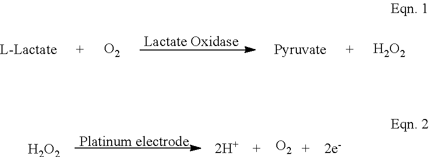

In the figure, LOD (ox) and LOD (red) refers to lactate oxidase in the oxidized and reduced forms respectively. Lactate oxidase is an enzyme that acts as a catalyst that may be immobilized on the platinum electrode. The blood lactate reacts with the oxygen in the presence of lactate oxidase and produces pyruvate and hydrogen peroxide as in Eqn. 1 above. When an appropriate voltage is applied, hydrogen peroxide disassociates on the surface of the electrode producing hydrogen ions and electrons as in Eqn. 2 above. Subsequently, the electrons are taken up by the working electrode, producing a current. The production of electrons is proportional to the amount of lactate thus making amperometric measurement of lactate possible. The enzyme lactate oxidase cycles between the oxidized and reduced forms within the immobilized layer as shown in the figure.

The amperometric measurement of glucose can be done in a similar manner. The chemical reactions are given below.

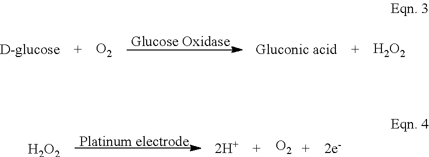

Hence reactions for the glucose measurement are similar to the reactions for the lactate measurements. The concepts described below apply equally to both these types of sensors.

The electrodes can be composed of a metallic or nonmetallic element, composition, alloy, or composite that is inert in vivo, including, by way of example: a metal per se, such as gold, platinum, silver, palladium, or the like; an alloy of two or more metals, e.g., a platinum-iridium alloy; a metal-coated substrate, such as a platinum-plated titanium or titanium dioxide substrate, or a platinum- and/or ruthenium-coated nickel substrate; a metal oxide, e.g., ruthenium oxide (i.e., ruthenium (IV) oxide, or RuO₂), rhenium oxide (generally rhenium (IV) oxide [ReO₂] or a composition of mixed-valence rhenium oxides), iridium oxide, or the like; a metal carbide such as tungsten carbide, silicon carbide, boron carbide, or titanium carbide; graphite; carbon-polymer composite materials, and combinations or mixtures of any of the foregoing. Electrodes of graphite, carbon-polymer composites, and noble metals are generally preferred. Noble metal electrodes include, for example, electrodes fabricated from gold, palladium, platinum, silver, iridium, platinum-iridium alloys, platinum-plated titanium, osmium, rhodium, ruthenium, and oxides and carbides thereof.

Carbon-polymer composite electrodes are fabricated from pastes of particulate carbon, e.g., carbon powder, carbon nanoparticles, carbon fibers, or the like, and a thermosetting polymer. Carbon-polymer composite electrodes are particularly desirable, for economic as well as practical reasons. Aside from the relatively low cost of such electrodes, use of a precursor composed of a paste of particulate carbon and a thermosetting or thermoplastic polymer or prepolymer thereof enables manufacture of the implantable catheter via extrusion, with the electrodes extruded along with the polymeric catheter body. Illustrative polymers for this purpose include, without limitation, polyurethanes, polyvinyl chloride, silicones, poly(styrene-butadiene-styrene), polyether-amide block copolymers, and the like. Carbon-polymer pastes for this purpose are readily available commercially, e.g., from ECM, LLC, in Delaware, Ohio. Preferred polymers are thermoplastic. Depending on the polymer system selected for electrode preparation, a polymerization initiator and cross-linking agent may be included in the fabrication mixture.

FIG. 1B illustrates the detail of the electrodes. The disassociation of hydrogen peroxide and the production of electrons occur at different voltages depending on the materials used for the electrodes. For example, for a carbon electrode, about 1V-2V is needed for the disassociation to take place. For a platinum electrode, the disassociation takes place at voltages typically less than 1V. However if voltages on the order of 1 V is applied to the electrodes, many species other than hydrogen peroxide will be oxidized such as ascorbic acid, uric acid, amino acids etc., all of which will produce erroneous currents. Hence for accurate measurement of hydrogen peroxide, the voltage needs to be reduced. With appropriate catalysts, the hydrogen peroxide oxidation is able to occur at very low voltages (under 0.5 V). One example of a catalyst that lowers the voltage requirement is 5% rhodium loaded carbon. Apart from the catalyst material being expensive, currently used manufacturing techniques also contributes to the cost as typically, the entire electrode is made of the expensive catalyst-loaded carbon. Hence in a departure from the typical manufacturing techniques, we developed devices to minimize the amount of catalyst. In one embodiment, the catalyst is provided as thin, micro-surface coating, on the order of microns, e.g. 1 or 10 to 100 or 1000 um) and is limited to the distal tip of the electrode that is exposed to blood, typically on the order of millimeters, e.g. 0.1, 0.2, 0.5 or 1 to 1, 2, 5 or 10 mm). In addition to the catalyst being used sparingly, another method to reduce cost of the electrodes is to use the catalyst only on one of electrodes where the chemical action predominantly takes place.

Referring to FIG. 1B now, a cross section of the electrode is illustrated with the various layers. Layer 16 is the substrate typically 50 um to 500 um thick. Materials for this layer are typically plastic, polyvinyl chloride (PVC), kapton (poly-4,4′-oxydiphenylene-pyromellitimide) and polycarbonate. Layer 17 may be a carbon layer typically in the range of 10 um-50 um thick. Layer 18 is the catalyst layer which may be less than 0.5 um thick. As stated before, typically in the current manufacturing techniques, layer 17 and layer 18 are combined. Finally layer 19 is the enzyme layer immobilized in a matrix such as an albumin matrix.

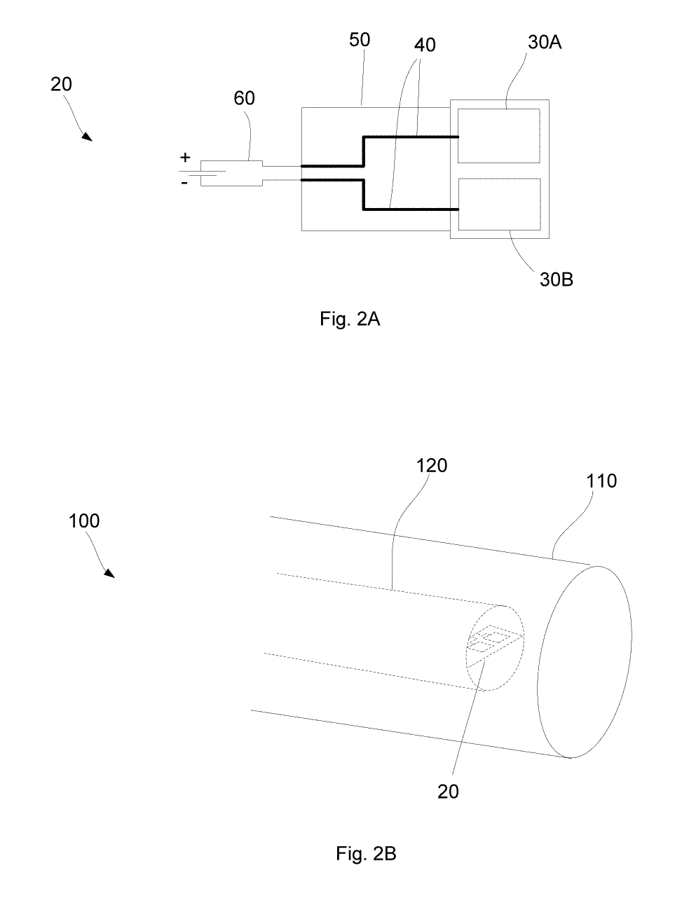

FIG. 2A describes a sensor 20 which may be utilized to measure blood lactate or glucose. The sensor 20 has two pads 30A and 30B which may form the working electrode and the counter electrode respectively. These electrodes may be made of several inert materials including but not limited to inert substances such as carbon, platinum, gold, palladium, alloys of two or more metals such as platinum-iridium etc. The sensor pads may be located on a substrate 50 which is an insulator material. The pads may be electrically connected via leads to the edge of the substrate which then is then subsequently connected to an external power source 60 such as a battery. The electrodes such as 30A and 30B are often referenced herein as “sensor electrodes”.

FIG. 2B describes how this sensor may be utilized. The sensor 20 may be placed at the distal end of a catheter 120 such as a central venous catheter (CVC). The catheter 120 and the sensor 20 are shown by dashed lines in the figure to indicate its placement inside a blood vessel 110. The catheter may be placed in various blood vessels including to the superior vena cava (SVC).

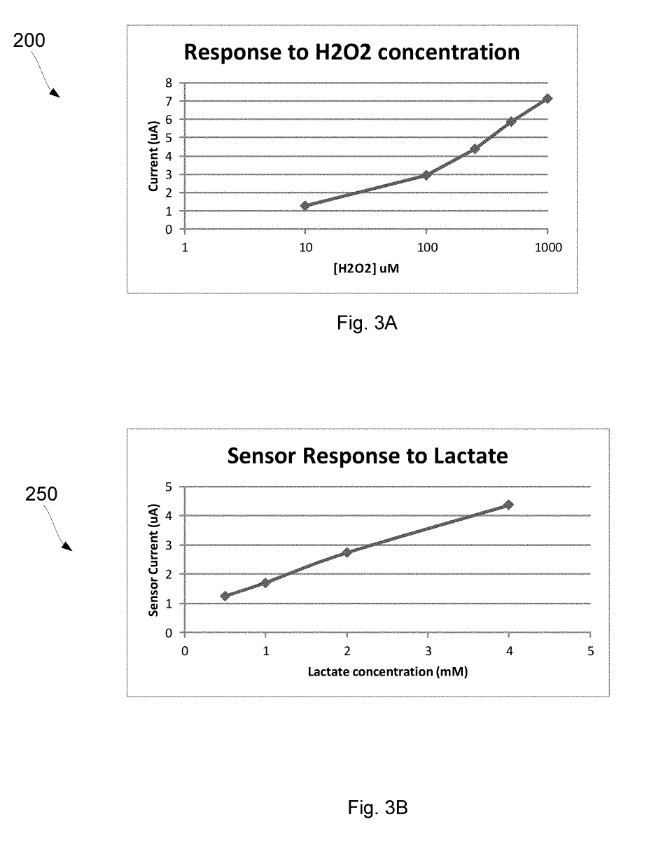

FIGS. 3A and 3B illustrate the observed response of a fabricated lactate sensor. While these graphs are instructive, the sensor characteristics and behavior may differ due to a variety of reasons, such as the exact sensor design. FIG. 3A is a graph illustrating the relationship between the observed current and the concentration of H₂O₂. FIG. 3B is a graph of the observed current and the concentration of lactate according to Eqn. 1 and Eqn. 2. The graph may be made linear in the lower concentration range or the upper concentration range using known techniques such as adjusting the level of enzyme immobilized or by controlling the level of lactate reaching the enzyme layer. The exact shapes of the graphs in FIGS. 3A and 3B are not important. However, the design of these sensors must be such that the relationship between the H₂O₂ concentration and subsequently the lactate concentration must be repeatable and monotonic. Once a repeatable relationship between current and lactate concentration is obtained, this relationship may be incorporated in a system comprising the catheter and the sensors (including other electronic components such as the power source) in various well known ways. For example, a portable digital current ammeter may be coupled to the circuit externally (i.e. outside the body of the patient) which may send its readings to a look up table (LUT). The LUT is a well-known way of relating two or more variables. The output of the LUT may be the lactate concentration.

Biofouling Immune Sensors

In measuring a blood analyte with the system outlined in FIGS. 2A and 2B, if the tip or the sensor become occluded by a blood clot, then the sensor readings may become faulty, leading to misdiagnosis and inappropriate treatment. The risk of blood clots is worse in situations where the catheter remains in the body for long periods of time. In addition to blood clots, the sensors may be susceptible to being infected with bacteria and fungi.

Anti-fouling did not expect anticlotting effect.

The invention provide devices, methods and systems to prevent blood clots from forming or if formed, to dissolve them without medication, and/or to treat or prevent infection. In brief, the method consists of laying down another set or sets of electrodes in the vicinity of the analyte sensors. When a current is passed through these additional set of electrodes, we found that blood clots tend to dissolve.

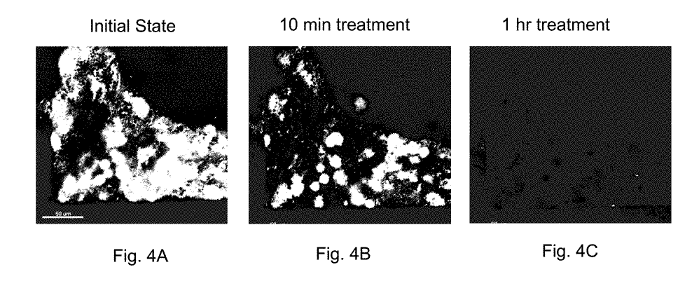

The additional set or sets of electrodes are referred to as biofouling prevention electrodes for the rest of this disclosure to distinguish them from the sensor or analyst sensing electrodes used for sensing blood compounds such as lactate and glucose. FIGS. 4A, 4B and 4C illustrate the results of in-vitro testing regarding blood clots. In these experiments, a flowing blood model was utilized where some blood coagulation was created on a conductive gold grid. FIG. 4A shows the initial state of the gold grid—the white sections of the figure show where the blood is clotted. In the initial state, there is no electrochemical activation, i.e. the control. The clots show up as a light color due to DNA staining of the blood. A current of 20 uA was then applied for 10 mins. The image of the clots (after the 10 minute application) on the gold grid is shown in FIG. 4B. From this figure, it is seen that the total area that is white in color is reduced. The same current was then applied for 1 hour and the results of this treatment are illustrated in FIG. 4C, where hardly any white areas are seen at all. This experiment demonstrated the surprising finding that applying a current can be effective in prevention or dissolution of clots.

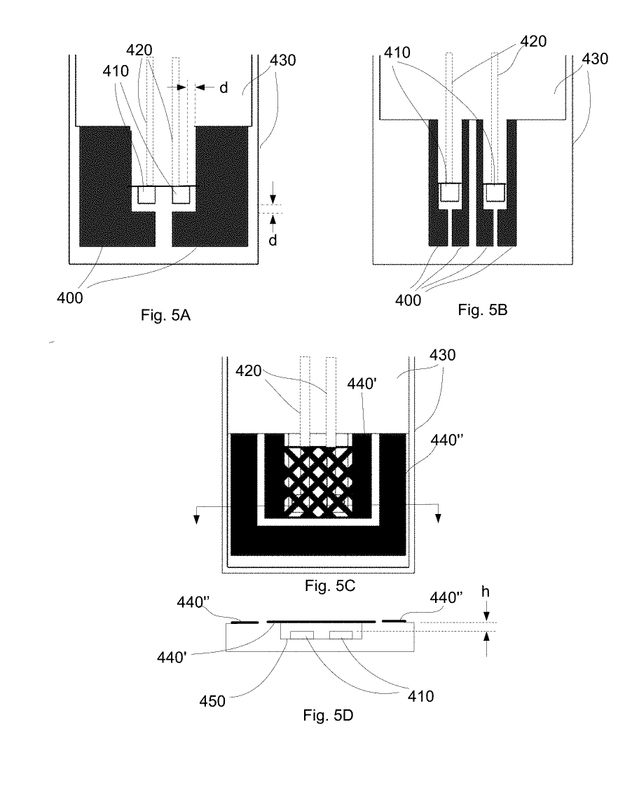

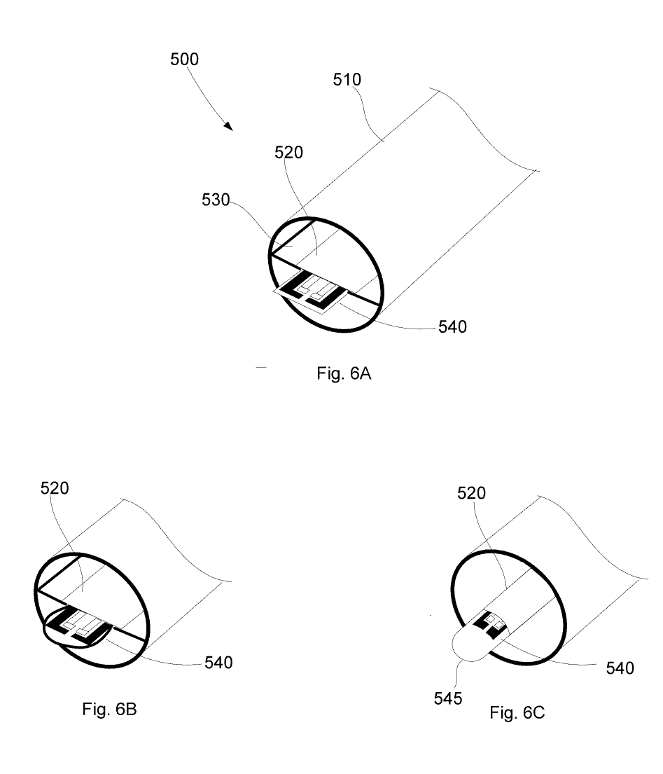

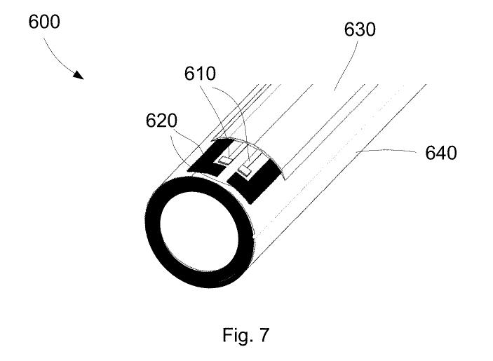

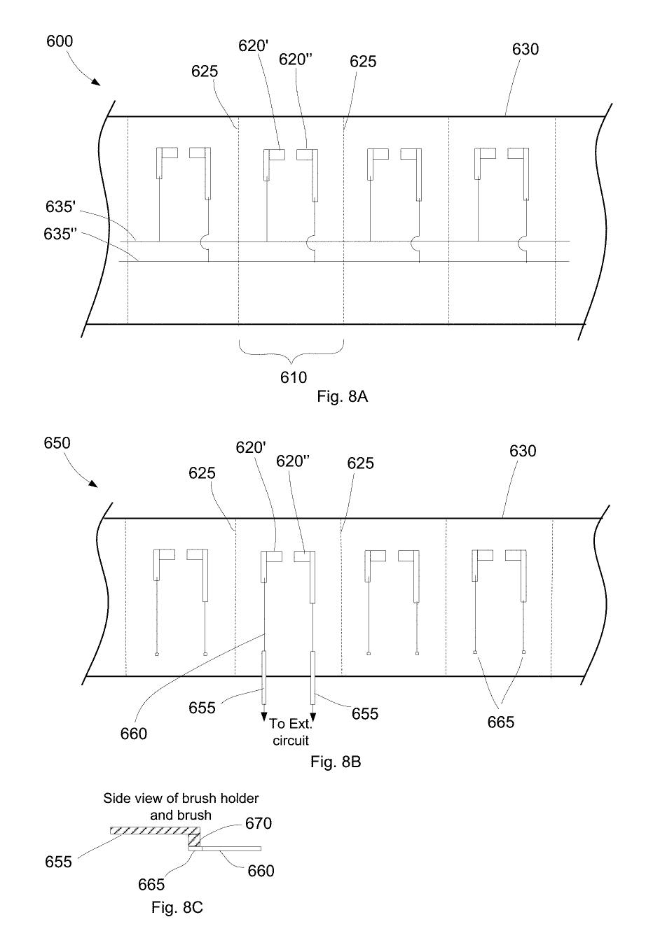

FIG. 5A-D illustrate different configurations of how the biofouling prevention electrodes may be arranged around the sensor electrodes. In these figures, 410 is the sensing electrode where the catalyst is placed and 420 refers to the leads which are normally under an insulator 430. In FIGS. 5A and 5B, 400 refers to the biofouling prevention electrodes whereas in FIGS. 5C and 5D, 440′ and 440″ refers to the same. FIG. 6A-C will illustrate how these configurations are placed within a catheter, however for now, focusing on FIGS. 5A-D, as shown in FIG. 5A, the biofouling prevention electrode is placed at a distance d from the sensors 410 both in the horizontal and vertical direction. Depending on the manufacturing processes, d can be as small as possible for example, 5, 10, 25, or 50 um to 100, 500, 1000 or 2000 um. As specified in WO2012/177807 the gap between the sensor electrodes may range from 0.1, 0.5 or 1 um to 50, 100 or 200 um.

FIG. 5B illustrates another configuration of the biofouling prevention electrodes and the sensor electrodes. Here each of the sensor electrodes is covered individually on either side by the biofouling prevention electrode.

FIG. 5C is a departure from the planar designs of FIGS. 5A and 5B. Here the sensor electrodes may sit in a depression of a pocket 450. The biofouling prevention electrodes are denoted by 440′ and 440″. Electrode 440′ as illustrated in the figure is a grid that allows blood plasma to pass through so that the lactate and glucose levels may be measured. The grid however blocks the other components of blood such as platelets, white blood cells, red blood cells from passing through so that blood does not clot. The anti-clotting behavior of the grid will prevent any clots from blocking the passage of plasma to the sensing electrodes. The openings of the grid may be of the order of less than 10 um preferably less than 5 um. White blood cells are typically 10-12 um whereas red blood cells are typically 7-8 um. The height h may be as small as possible according to manufacturing techniques but preferably less than 50 um. It may be larger than 50 um hence no limitation is intended. In addition to blocking the white blood cells from entering the pocket, the electrochemical activation between the electrodes 440′ and 440″ may also prevent biofilms and blood clots from forming in the vicinity of the sensor so that the measurements of blood components such as glucose and lactate are not hampered.

FIGS. 5A-5D describe in detail how a sensor may be shielded from clots or from biofilms with biofouling prevention electrodes. FIG. 6A illustrates how such a sensor may be coupled with a catheter. In this figure a bi-lumen catheter 510 is illustrated although the catheter may have a single lumen or have more than two lumens. The lumen wall inside the catheter is shown as 530. The sensor 540 and the insulator 520 may be slid into one of the lumens after catheter placement into the body or permanently attached into one of the lumens (i.e., the catheter and the sensor may be manufactured as one integral unit). Although in the FIGS. 5A-5D and 6A, the sensors are shown having a rectangular format, it may be advantageous to have other shapes that make the sensor atraumatic. In other words, a sensor having a sharp edge or a corner may promote the formation of blood clots. In order to minimize or remove the possibility of blood clot formation, various techniques are now described. In one technique, the edges of the sensor may be rounded or coated with substances such as but not limited to hydrogel. In another technique illustrated in FIG. 6B, the sensor base may have a shape that does not have any sharp corners such as but not limited to a circular shape or an oval shape. In yet another technique, the sensors electrodes and the biofouling prevention electrodes may be coupled on to a rod as shown in FIG. 6C. In yet more techniques, a tear drop shaped three dimensional structure or two thinner rods (such as shown in FIG. 6C) may be utilized. In the case where two thinner rods are utilized, each rob may have its own electrode. The latter configurations are not shown in the figure.

In some circumstances, the length between the distal end and the proximal end of the catheters may be quite large in the order of 10 cm-15 cm in the case of a CVC. Since the sensor electrodes need to be sensitive to very small currents (microAmps as illustrated in FIGS. 3A and 3B), the transmission of these currents over the cable length needs to occur in a reliable and noise free manner. One method to achieve the transmission is to match the impedance of the electrodes and the cables by transforming the impedance of the sensor to a lower value using an impedance transforming integrated circuit chip. Other methods may include amplifying the currents and sending the amplified currents. Yet other methods may include digitizing the signals in the near vicinity of the sensors and transmitting digital signals. These are all well-known methods of driving small signals over a cable. However, common to all these methods is that there needs to be a miniaturized electronics module in the near vicinity of the sensor electrodes. The electronics module may be coupled to the insulator substrate and may be as simple as a resistor-inductor-capacitor (RLC) network. Other electronics components may include but not limited to filters and analog-to-digital chips.

In some other concepts, the authors have observed that the destruction and removal of biofilms and thrombus is greater at the working electrode. Thus, based on this observation, the polarity of the electrodes may be switched periodically. The switching period may be in the order of minutes for example 15 minutes. The switching periods do not have to be accurate from cycle to cycle; hence an inexpensive method may be chosen to cause the switching. For example a double pole, double throw (DPDT) low voltage relay may be used to achieve switching. In some other concepts an alternating current may be used to switch the polarities.

WO2012/177807 described methods and systems to prevent biofouling of implantable catheters. In that disclosure, electrodes were placed on the outside and inside surfaces of the catheter. In a further concept, sensor electrodes may be coupled on the surfaces of the catheter in addition to having the biofouling prevention electrodes in the near vicinity. This concept is described in FIG. 7. In this figure, 640 is a catheter upon which sensor electrodes 610 may be coupled. In the near vicinity of the sensor electrodes, the biofouling prevention electrodes are coupled to the catheter 640 as illustrated in the figure. Except at the very distal tip, the electrodes are covered by the insulator layer 630. Materials and methods of manufacture of the catheter and the biofouling prevention electrodes are described in WO2012/177807. The sensor electrodes may be made of several materials including but not limited to platinum. The methods of laying down these electrodes are much the same as for the biofouling prevention electrodes.

In another concept, the biofouling prevention electrodes may only apply the voltages necessary for removal of blood clots when it senses that a clot has formed across the sensor surface. For example, referring to FIGS. 5A-5C, a low voltage of 10-50 mV may be continuously applied across the electrodes 400 (the biofouling prevention electrodes). The impedance across the electrodes may be continuously monitored from the instant the sensor is immersed in an environment where blood chemical monitoring is required. Initially, when the sensor is immersed in blood and assuming no clots are formed, the impedance across the electrodes 400 is expected to be low, such as between 0 and a few milliohm, though other values are not excluded. However, as a blood clot forms, the impedance is expected to rise significantly. An external circuit may monitor the impedance and at some threshold level for example if it sees a 50% increase in impedance, the external circuit may trigger the application of much higher voltages for example 1V-2V across the electrodes 400. Thus in this system, a low voltage is always applied to the biofouling prevention electrodes but then when a clot is detected, higher voltages are applied to dissolve the clot.

In an extension of this concept, the response of the sensor electrodes may be modulated depending on the sensed impedance. As clots form, the measured concentration of blood chemicals such as lactate and glucose may vary; for example the impedance may go down. A system which would comprise the sensor electrodes and the biofouling electrodes may also contain a value adjusting function such that depending on the sensed impedance as measured across the biofouling prevention electrodes, the values of the concentration of the blood chemicals as measured by the sensor electrodes may be modulated such as multiplied by a certain factor. Prior calibration would be required to associate a multiplicative factor to a value of the sensed impedance. These calibration values may be stored in a processor or a look up table (LUT) which would be part of the system mentioned above.

Biofouling Immune Sensors without Electrodes

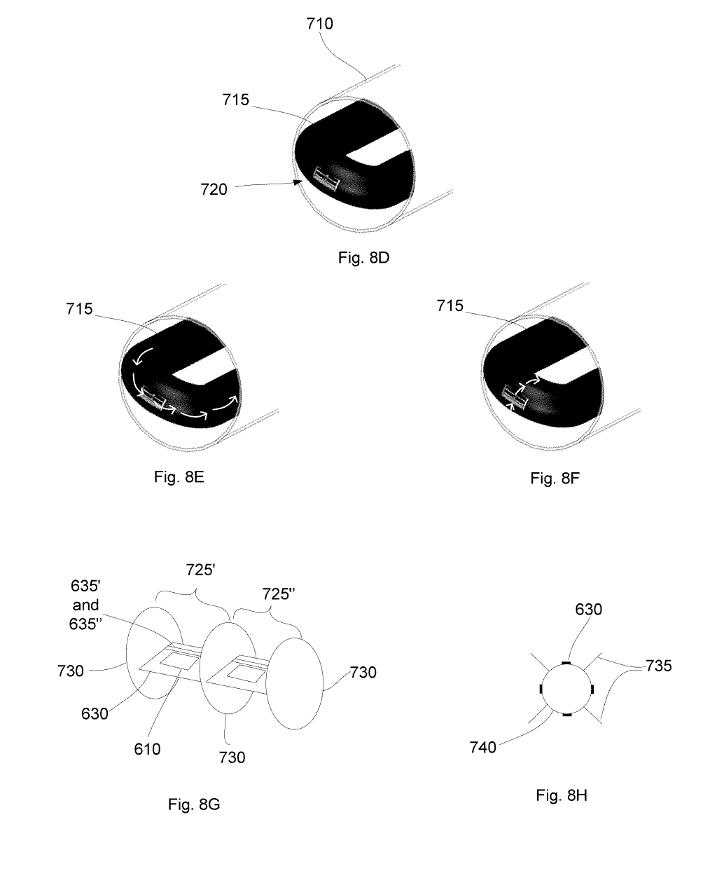

In the concepts above, the sensors were surrounded by biofouling prevention electrodes either in a planar manner or in a non-planar manner. In further concepts described below, biofouling prevention is obtained without the use of biofouling prevention electrodes. FIG. 8A-8H explains these concepts. The concepts are based on having a series of sensors printed on a strip which can be rotated within a catheter so that each sensor is exposed blood only once for a short amount of time such as less than 5 mins. Other exposure times are not excluded. By exposing each sensor only once, the risk of biofouling including the buildup of bacteria, fungi and thrombus is significantly curtailed or eliminated. FIG. 8A illustrates a strip of sensors. The strip base is shown as 630 which may be made of an insulator material. The strip base may be made in sections such as 610 which may be stiff and does not bend so that the electrodes may be supported by it. Each section may be separated from the next by a bendable section indicated by dashed lines 625. The strip base 630 may be made of some biocompatible material such as polyvinylchloride (PVC). The bends or the folds may be created by one of several well-known methods such as scoring or laser cutting a groove or channel on the strip base material. The electrodes may be coupled to the strip base using one of various well known methods such as deposition, printing etc. 620′ and 620″ are sensor electrodes. 635′, 635″ are electric lines carrying voltage and current to these sensors. The sensors are attached in parallel to these lines. Thus, all the sensor electrodes are attached in parallel to lines 635′ and 635″. Each section such as section 610 may also have an electronics module to achieve impedance matching as explained earlier. The electronics module is not shown in the figure.

An alternative design for the arrangement shown in 600 is shown in FIG. 8B. In this figure, the strip base and electrodes are laid out similarly as in 600. The difference is that the electrical connections are made via brushes which stay stationary in one place while the strip slides beneath it. The brush holders are shown as 655. FIG. 8C shows the side view of the brush holder, the brush 670 which is immovably coupled to brush holder. Each electrode has electrical connections such as 660 with pads at the end shown as 665. When the pads 665 are directly below the brush 670, electrical connections are made. Thus only one set of sensor electrodes is active when its pads such as 665 are directly below the brushes 670.

FIG. 8D shows how such a strip of sensors may be utilized in a catheter. The catheter is shown as 710 inside which exists another sheath 715. The sheath 715 has an opening 720 which may allow blood to go through. Inside the sheath, the sensors would pass by one at a time below the opening 720. A mechanism at the proximal end will cause the sensors to move inside the sheath. As each sensor passes through the opening 720, that specific sensor may come in contact with blood and the lactate or glucose levels may be measured by that sensor. In the scheme 600 in FIG. 8A all sensors are active but only one sensor which is in contact with blood will provide the measurement. In the scheme 650 of FIG. 8B, only one sensor (the sensor with its pads under the brush) is active and will provide the measurement.

FIGS. 8E and 8F show the various ways the sensors may be arranged to slide under the opening 710. In FIG. 8E, the white arrows indicate that the sensors slide along the length of the inner sheath 715. In FIG. 8F, the white arrows indicate that the sensors rotate in a circular fashion inside the sheath. In FIG. 8F, the sensors may be mounted on the length of a drum which may be mounted within the sheath. The circular drum can be caused to rotate by torquable wire which may be mounted at the center of the drum and activated by motors outside the body.

To prevent the blood from coming in contact with more than one sensor at a time, each sensor may be compartmentalized. Thus for configuration 600, the compartmentalized sensors are illustrated in FIG. 8G. Two compartments 725′ and 725″ are shown although there may be more. Each compartment may have two walls indicated by 730 which may be made of the same insulator material as the strip base 630 such as but not limited to PVC. The electric lines are shown for each compartment. In this case, the configuration 600 shown in FIG. 8A is illustrated. The lines may pierce each wall so that a continuous line may be achieved. The conduits where the lines pierce the walls may be sealed so that blood does not leak through. FIG. 8H describes how the arrangement in FIG. 8F can be compartmentalized. Here, 740 is the drum upon which four sensors such as 630 are shown arranged along the length of the drum. Walls 735 may be arranged along the length of the drum so that any blood that seeps through the opening 720 stays within the compartment. The diameter of the drum and the walls may be just slightly smaller than the inner diameter of the sheath 715 so that all the blood remains within the compartment but the arrangement may rotate within the sheath.

If the arrangement with the brushes is used as seen in 650, then returning back to FIG. 8G, the walls 730 may have electrical connections that run along a radius of the wall as shown in FIG. 8I. Only one wall is shown for convenience. The wall in this figure is shown to have some thickness which is indicated by 750. The sensor strip 630 is shown with one sensor and with electric lines 660. The electric lines may continue along the circular wall as shown in 745. Lines 745 may then run along the thickness dimension of the wall where it may then connect to a brush which would be placed on the inside surface of the sheath 715.

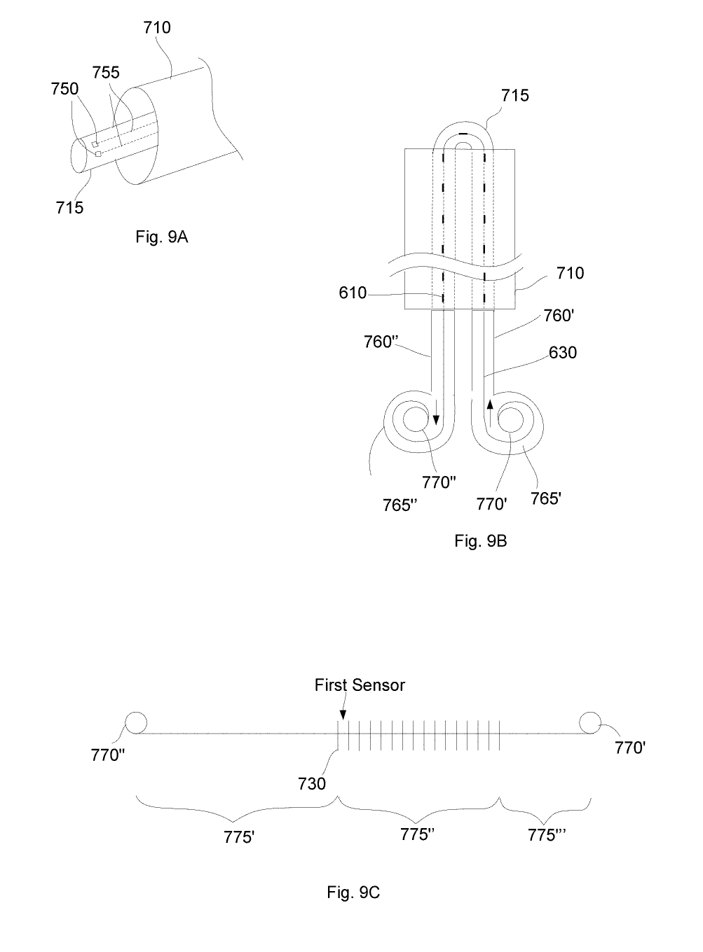

The proximal ends of the catheters with sensors as described in FIGS. 6A-C or as described in FIGS. 8A-I will depend on which type of sensor is chosen. If the design in FIG. 6A-C is chosen, the electrical wires may exit the proximal end and may be connected to pads that are embedded in the walls of sheath in such a manner that there is no entry or exit for materials such as blood or for microorganisms. The risk of infection is then reduced or minimized. This arrangement is shown in FIG. 9A. In this figure, the proximal end of catheter 710 is illustrated. The sheath 715 exits the proximal end. The wires 755 are on the inside of the sheath hence they are shown by dashed lines. Connections 750 are inset with the body of the sheath 750 and are sealed in such a manner that no bodily fluids can come out and no microorganisms can enter the sheath.

FIG. 9B illustrates detail of the proximal side and a section of the distal side of a catheter and sensor configuration if a strip of sensors such as illustrated in FIG. 8A or FIG. 8B is utilized. The components inside the catheter are shown by dashed lines. Here, since the strip is being utilized, a single sheath 715 that is doubled back at the distal end is utilized. 760′ and 760″ illustrate the two ends of this sheath at the proximal side. Each end of the sheath is coupled to an out-take and an in-take holders 765′ and 765″. These holders provide space for containing the strip of sensors 630 in a sterile manner both before entry into the catheter 710 and after exit from the catheter 710. 770′ and 770″ are cylinders that can be driven by external motors that are coupled to the end of the strip. The sensor sections are illustrated by 610. Only one sensor section is enumerated. Cylinder 770′ can rotate clockwise and feed the sensors into one end of the sheath (760′) while cylinder 770″ can also rotate clockwise but roll up the used sensors upon themselves. The electrical connections can be made just as explained in FIG. 9A and can be on any one of sides of the sheath 760′ or 760″. Thus by controlling the two drums 770′ and 770″ a new sensor may be exposed to blood under the opening 720 as shown in FIG. 8E. To accommodate the compartments shown in FIG. 8G if they are needed and used, some sections of the strip may not have sensors and compartments. For example, at the start of the procedure, the first sensor may be already placed at the opening. Sensors may be laid out from the first sensor up to where the end of the sheath 760′ occurs and where 765′ begins. The section of the strip inside 765′ may not have any sensors or compartments. It just provides the feed for sensor strip. Then on the other side from the first sensor to drum 770″, no sensors or compartments may be laid out. Here the strip simply provides mechanical continuity so that when 770′ and 770″ roll in a clockwise manner, sensors are exposed one at a time to blood. The configuration of the strip is illustrated in FIG. 9C.

In FIG. 9C, three sections 775′, 775″ and 775′″ are illustrated. Section 775′ has no sensors and is coupled to the in-take drum 770″. The second section 775″ has the compartments separated by the walls 730. Only one wall is labeled. The sensors are laid out between the walls. The location of the first sensor is indicated. Before first use, the first sensor is placed under the opening. The third section 775′″ again has no sensors and is coupled to the out-take drum 770′. Although not obvious, the section 775′″ continues in around the drum 770′ so that as the drums rotate in a clockwise manner, drum 770′ lets out more of the section 775′″ and drum 770″ takes up the section 775′. Thus with this arrangement, the sensors may be exposed one at a time to blood and can accommodate the compartments.

Although the concepts above describe compartmentalized sensors when a strip sensor is used, it may be found in practice that the compartments may not be needed at all. Blood may reach the sensors and it may clot over the sensor after a certain time. A new sensor will need to be exposed if a new measurement is required. All the concepts above are still relevant except the walls 730 may not be needed if there is no chance of contamination of the unexposed sensors.

In a variation of the strip of sensors concept, the strip may contain two or more different types of sensors, each for a different analyte, such as lactate, glucose or urea sensors, in alternating manners. In a further variation of this concept, an identification system is included in the design such that the sensor type that is currently active can be identified. Knowledge of the type of sensor that is active then enables the identification of the blood chemical being sensed or measured. Identification of the type of sensor may be done using various methods. In one method, the electronics module described earlier may be used. As described earlier, the electronics module conditions the sensor signal by impedance matching or amplification so that the small currents can be detected. The need for impedance matching and amplification arises because the small currents have to be carried by relatively long wires to other electronic components outside the body. However, the electronics module may include another component which imparts a specific characteristic to a signal so that later in the circuit, the characteristic can be used to know which type of sensor is making the measurements. As an example, if an analog-to-digital converter chip is within the electronics module, each sensor may have a signature binary code which may be sent just before the sensor starts to measure the blood chemicals. The processor (typically located outside the body) would recognize the code and will know the type of sensor the information the processor receives subsequently to receiving the code came from. Thus sensor identification may be carried out. Other methods may also be used for sensor identification.

FIGS. 8A through 8H and 9A through 9C thus explain the various ways sensor strips with no biofouling prevention electrodes may be used to measure components of blood such as lactate and glucose.

In Vitro Series Measurement of Blood Compounds

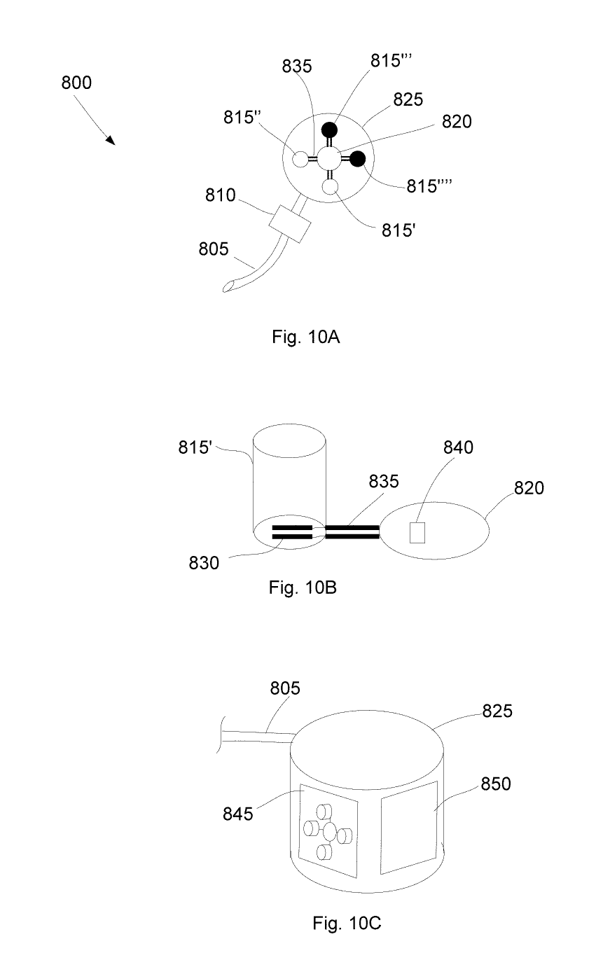

FIG. 10A-C illustrate another concept of making a series of measurements of blood compounds such as lactate or glucose but making these measurements in-vitro. In FIG. 10A, 805 is a catheter which may be inserted into a vein for the period that measurements need to be taken. This catheter may be preferably of 1 cm in length although other lengths are not excluded. A micro-pump 810 is included in the figure to illustrate that if capillary action is not enough to draw blood, a micro-pump may be added to promote flow. Hence either through capillary action or through the actions of the micro-pump, small quantities of blood is drawn from the body and is deposited into empty containers which form part of the microanalysis platform 825. The top view of the microanalysis platform is shown in FIG. 10A. It is shown with the cover off. It contains four hollow containers or cylinders 815′, 815″, 815′″ and 815″″ although there may be more or less cylinders. The micro-pump draws blood which fills these cylinders one at a time. The amount of blood needed per cylinder may be very small such as less than 2 ul. Two of the cylinders are shown black as they indicate these cylinders are already filled with blood. FIG. 10B shows an empty cylinder such as 815′ so that some features can be explained in more detail. The cylinder as explained above receives blood. The cylinder has two sensor electrodes such as those shown in FIG. 2A. These sensors measure the lactate or the glucose levels amperometrically. No biofouling prevention electrodes are needed as each cylinder is used once and never used again. The cylinders are all mechanically supported via support booms 835 which may also provide voltage to the electrodes. Finally a central platform 820 houses electronics and batteries for the electrodes in addition to providing mechanical support for the booms. The central platform may be supported on its axis by a rotating spindle which may be attached to a small motor. Thus, depending on the timing circuit which may be housed on the central platform, micro-pump may be activated at a certain time when an empty cylinder in placed in a position so that it can receive blood and do the analysis. The motor and the power supply for the motor may be located below the central platform but enclosed within the housing of the microanalysis platform.

FIG. 10C provides a perspective view of the microanalysis platform 825. In this case, the cylinders are mounted vertically on the side of platform. The actual positioning of the cylinders is not critical as long as it is able to receive the blood from the micro-pump. In this figure, a small liquid crystal display (LCD) screen 850 is provided which may be utilized to output the sensor values or other information. An advantage of this type of system is that blood used for analysis is never returned to the blood supply; hence biocompatibility is not an issue. In addition, biofouling prevention may not be needed for this system.

Various configurations have been provided to measure levels of blood compounds such as lactate and glucose. Some of these configurations describe methods and systems to prevent biofouling. Some other configurations are provided that do not have biofouling prevention but they solve the issue of biofouling in a different manner. Finally some configurations are able to make measurements in-vivo whereas some configurations make these measurements in-vitro.

### REFERENCES

[1]. An evaluation of serial blood lactate measurements as an early predictor of shock and its outcome in patients of trauma or sepsis by U. Krishna et. al. Indian Journal of Critical Care Medicine 2008 April-June: 2013, pp 66-73.
[2] Management of occlusion and thrombosis associated with long-term indwelling central venous catheters by Jacquelyn L. Baskin et. al, Lancet, 2009 Jul. 11.
[3] The need for continuous blood glucose monitoring in the intensive care unit by ram Weiss et. al, Journal of Diabetes Science and Technology, Vol 1, Issue 3, May 2007

The invention encompasses all combinations of recited particular and preferred embodiments. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

What is claimed is:

1. A device configured to provide serial electrochemical measurements of blood components, the device comprising:
a pair of anode and cathode elongate sensor electrodes, each comprising a distal, terminal tip comprising a surface catalyst which catalyzes a chemical reduction-oxidation (redox) reaction of a blood analyte yielding an amperometric measurement of the analyte; and
a pair of anode and cathode elongate antifouling electrodes, each comprising an uninsulated, distal, terminal tip, between which an electrical current flows,
wherein the sensor and antifouling electrode tips are disposed on a planar surface, which may be flat or curved, and the antifouling electrode tips sufficiently surround one or both of the sensor electrode tips wherein when disposed in a vein the current causes chemical reactions in the blood around one or both of the sensor electrodes tips which reduces or prevents biofouling of the tip of one or both of the sensor electrodes.

2. The device of claim 1 wherein:
the planar surface is flat;
the sensor electrode tips are disposed on insulator pads;
the device is disposed in the lumen of a vein or artery;
the device is disposed in the lumen or on the surface of an implanted catheter;
the sensor electrode tips are separated by 1 nm to 1 mm;
the sensor and antifouling electrode tips are separated by 1 nm to 1 mm;
the biofouling prevention electrodes are at a distance from the sensor electrodes about or between 1000 um and 1 um;
the sensor electrodes are set in a pocket covered by one of the antifouling electrode tips patterned as a grid; and/or
the sensor electrodes are set in a pocket covered by a first of the biofouling prevention electrodes and patterned as a grid with hole size smaller than the size of white blood cells.

3. The device of claim 1 configured for:
switching the polarity of the biofouling prevention electrodes;
measuring an analyte that is lactate or glucose; and/or
preventing biofouling that is tissue or particle deposition, such as resulting from clot formation.

4. A method of using the device of claim 1 comprising (a) continually or continuously measuring impedance between the biofouling prevention electrodes and switching on higher voltages when higher impedance is sensed; or (b) multiplying the concentration values read by the sensor electrodes by a constant dependent on the impedance between the biofouling prevention electrodes.

5. A device configured to provide serial electrochemical measurements of blood components, the device comprising:
a series of sensors, each sensor comprising a pair of elongate sensor electrodes, each comprising a distal, terminal tip comprising a surface catalyst which catalyzes a chemical reduction-oxidation (redox) reaction of a blood analyte yielding an amperometric measurement of the analyte at intervals, wherein the series is configured so that each of the sensors is exposed to blood for a predetermined time sufficiently limited to reduce or prevent biofouling of the tip of one or both of the sensor electrodes.

6. The device of claim 5, wherein the series of sensors is:
(a) printed on a rotatable strip within a catheter, rotated so that each of the sensors is exposed to blood for a predetermined time sufficiently limited to reduce or prevent biofouling of the tip of one or both of the sensor electrodes, wherein electrical connections to the tips are optionally made via brushes which stay stationary in one place while the strip slides beneath it;
(b) arranged on a strip configured to be inserted into a sheath with an opening such that only one of the series of sensors is exposed to blood at any one time;
(c) arranged on a side of a drum which rotates inside a sheath with an opening such that only one of the series of sensors is exposed to blood at any one time; or
(d) compartmentalized to prevent the blood from coming in contact with more than one of the sensors at a time,

7. The device of claim 5 wherein:
the sheath and the sensor strip are inserted into a catheter;
the sensors are arranged in a parallel configuration;
the sensors are arranged so that they come in contact with a brush placed inside the sheath;
each sensor is compartmentalized so that only the sensors not under the opening of the sheath are not contaminated; and/or
comprising multiple different analyte sensors in a single strip, such as lactate and glucose.

8. A method of using the device of claim 5 comprising taking with the device serial electrochemical measurements of blood components, wherein each of the sensors is exposed to blood for a predetermined time sufficiently limited to reduce or prevent biofouling of the tip of one or both of the sensor electrodes.
